(19) United States
(12) Patent Application Publication
(10) Pub. No.: US 2017/0307725 A1
(43) Pub. Date: Oct. 26, 2017

(54) METHOD FOR REDUCING SIDELOBE IN ULTRASOUND IMAGES

(71) Applicants: WAYGENCE CO., LTD, Chuncheon-si, Gangwon-do (KR); Daejin University Center for Educational Industrial Cooperation, Pocheon-si, Gyeonggi-do (KR)

(72) Inventors: Mok Kun JEONG, Seoul (KR); Sung Jae KWON, Seoul (KR)

(73) Assignees: WAYGENCE CO., LTD, Chuncheon-si, Gangwon-do (KR); Daejin University Center for Educational Industrial Cooperation, Pocheon-si, Gyeonggi-do (KR)

(21) Appl. No.: 15/515,690

(22) PCT Filed: Jan. 8, 2015
(86) PCT No.: PCT/KR2015/000165

§ 371 Date: Mar. 30, 2017

(30) Foreign Application Priority Data
Oct. 14, 2014 (KR) 10-2014-0138034

(51) Int. Cl. G01S 7/28; A61B 8/00; B06B 1/06

(52) U.S. Cl. CPC G01S 7/2813; B06B 1/0648; A61B 8/461; B06B 1/06

(57) ABSTRACT

According to the present invention, a method for reducing a sidelobe in an ultrasound image includes Step 1 of receiving, from individual receiving elements of an array transducer, ultrasonic signals reflected from an imaging point, and outputting the ultrasonic signals as channel signals of the corresponding receiving elements; Step 2 of applying focusing delays to each of the channel signals to temporally align the channel signals; and Step 3 of synthesizing an ultrasound image by using an added-up signal which is obtained by adding up the temporally aligned channel signals, wherein Step 3 includes calculating a magnitude of a corresponding sidelobe signal by using a spatial frequency of the sidelobe signal which generates a sidelobe and the number of receiving elements and synthesizing the ultrasound image by subtracting the calculated magnitude of the sidelobe signal from the added-up signal.

### TECHNICAL FIELD

The present invention relates to a method for reducing a sidelobe in an ultrasound image, and more particularly, to a method for reducing a sidelobe in an ultrasound image in which a magnitude of a sidelobe signal which generates a sidelobe is calculated using channel signals obtained by applying focusing delays to signals received by an array transducer having a plurality of receiving elements, and an influence from the sidelobe signal is reduced by removing sidelobe signal components included in the channel signals by subtracting the calculated magnitude of the sidelobe signal in a process of adding up the delayed channel signals for focusing or by assigning a weighting value to an ultrasound image using the calculated magnitude of the sidelobe signal when synthesizing the ultrasound image, thereby improving image quality of an ultrasound image.

### BACKGROUND ART

Generally, ultrasound images are used in diagnosing lesions and are formed by transmitting ultrasonic signals by a transducer and then converting the magnitude of ultrasonic signals received after being reflected from an inside of a human body into brightness.

Despite the advantages of safety and real-time imaging capability, the ultrasound images have a low resolution problem compared to other medical images. To solve this problem, a method of using an array transducer to focus ultrasound waves of short pulse widths in order to transmit and receive the ultrasound waves is being applied in a general medical ultrasound imaging system.

Taking a close look at a sound field in the ultrasonic wave focusing system, the sound field has a characteristic in which a mainlobe is formed with respect to a scan line of a transducer and sidelobes are formed at both sides of the mainlobe due to leakage of ultrasonic signals. When sidelobes are formed in this way, because signals of a reflector in the direction of the sidelobes are also received, there is a problem in that the signals act as noise in ultrasound images and lower the resolution of the ultrasound images.

Consequently, various attempts for reducing the sidelobes in ultrasound images are recently being made, and details thereof are disclosed in detail in [Document 1], [Document 2] below.

However, in the cases of [Document 1] and [Document 2] below, since a method of applying weighting values to each received channel data is used, there is a problem of having to perform excessive calculations to reduce sidelobes, and this problem is aggravated further as the number of channels increases.

[Document 1] Korean Unexamined Patent Application Publication No. 2009-0042152 (published on Apr. 29, 2009)

[Document 2] Korean Registered Patent No. 971433 (announced on Jul. 14, 2010)

### DISCLOSURE

### Technical Problem

The present invention has been devised to solve the above-mentioned problems of the related art, and it is an aspect of the present invention to provide a method for reducing a sidelobe in an ultrasound image in which, after obtaining channel signals by applying focusing delays to signals received by an array transducer having a plurality of receiving elements, an already known spatial frequency of a sidelobe signal which generates a sidelobe and the number of channels of the array transducer are used to calculate the magnitude of the corresponding sidelobe signal, and sidelobe signal components included in the channel signals are removed by subtracting the calculated magnitude of the sidelobe signal in a process of adding up the focusing-delayed channel signals, thereby improving image quality of an ultrasound image.

It is another aspect of the present invention to provide a method of reducing a sidelobe in an ultrasound image in which, when synthesizing an ultrasound image using channel signals obtained by applying focusing delays to signals received by the array transducer, an influence from the sidelobe signal is reduced by assigning a weighting value to the calculated magnitude of the sidelobe signal, thereby improving image quality of an ultrasound image

### Technical Solution

To achieve the above aspects, a method for reducing a sidelobe in an ultrasound image according to the present invention includes Step 1 of receiving, from individual receiving elements of an array transducer, ultrasonic signals reflected from an imaging point, and outputting the ultrasonic signals as channel signals of the corresponding receiving elements;

Step 2 of applying focusing delays to each of the channel signals to temporally align the channel signals; and Step 3 of synthesizing an ultrasound image by using an added-up signal which is obtained by adding up the temporally aligned channel signals, wherein Step 3 includes calculating a magnitude of a corresponding sidelobe signal in channel signals by using a spatial frequency of the sidelobe signal which generates a sidelobe and the number of receiving elements and synthesizing the sidelobe suppressed ultrasound image by subtracting the calculated magnitude of the sidelobe signal from the added-up signal.

The calculating of the magnitude of the sidelobe signal in the temporally aligned channel signals may include Step 3-1 of extending a signal length of the sidelobe signal according to a predetermined method and Step 3-2 of estimating a waveform of the sidelobe signal having the extended signal length to calculate a frequency component of the corresponding sidelobe signal.

The sidelobe signal may be a signal whose spatial frequency is (positive integer+0.5) cycles per aperture (CPA) in an original channel signal, and Step 3-1 may include extending a signal length of the corresponding sidelobe signal by appending zeros to a front end, a rear end, or both of the corresponding sidelobe signal so that the spatial frequency becomes a nearest positive integer in an extended length signal.

A method for reducing a sidelobe in an ultrasound image according to the present invention includes Step 1 of receiving, from individual receiving elements of an array transducer, ultrasonic signals reflected from an imaging point, and outputting the ultrasonic signals as channel signals of the corresponding receiving elements; Step 2 of applying focusing delays to each of the channel signals to temporally align the channel signals; and Step 3 of synthesizing an ultrasound image by using an added-up signal which is obtained by adding up the temporally aligned channel signals, wherein Step 3 includes calculating a magnitude of a corresponding sidelobe signal by using a spatial frequency of the sidelobe signal which generates a sidelobe and the number of receiving elements and synthesizing the ultrasound image so that a brightness value of the ultrasound image is inversely proportional to the calculated magnitude of the sidelobe signal.

The magnitude of the sidelobe signal may be a quality factor (QF) value which is obtained by adding up magnitudes of a plurality of sidelobe signals having different spatial frequencies, and Step 3 may include synthesizing the ultrasound image so that the brightness value of the ultrasound image is inversely proportional to the QF value.

### Advantageous Effects

As above, a method for reducing a sidelobe in an ultrasound image according to the present invention is configured such that, after obtaining channel signals by applying focusing delays to signals received by an array transducer having a plurality of receiving elements, an already known spatial frequency of a sidelobe signal which generates a sidelobe and number of channels are used to calculate the magnitude of the corresponding sidelobe signal, and sidelobe signal components included in the channel signals are removed by subtracting the calculated magnitude of the sidelobe signal in a process of adding up the focusing-delayed channel signals. In this way, compared to the related art, there is an advantage of being able to easily improve image quality of an ultrasound image because a magnitude of a sidelobe signal that affects image quality of an ultrasound image can be accurately calculated even with a small amount of calculation and the calculation result is applied to synthesis of the ultrasound image.

### DESCRIPTION OF DRAWINGS

FIG. 1 is a block diagram illustrating a configuration of an apparatus to which a method for reducing a sidelobe in an ultrasound image according to an embodiment of the present invention is applied.

FIG. 2 is a view illustrating a sound field characteristic of a general ultrasonic wave focusing system.

FIG. 3 is a view for describing a principle of a sidelobe formation in a sound field of an ultrasonic wave focusing system.

FIG. 4 is a view illustrating a waveform of signals across a transducer, which impinge on the transducer at a certain angle shown in FIG. 2.

FIGS. 5 and 6 are views for describing a calculation process for removing a sidelobe by an apparatus for reducing a sidelobe in an ultrasound image according to an embodiment of the present invention.

FIGS. 7 to 9 are views illustrating test results for comparing advantageous effects of the method for reducing a sidelobe in an ultrasound image according to the present invention with those of the related art.

### MODES OF THE INVENTION

Hereinafter, embodiments according to the present invention will be described in detail with reference to the accompanying drawings.

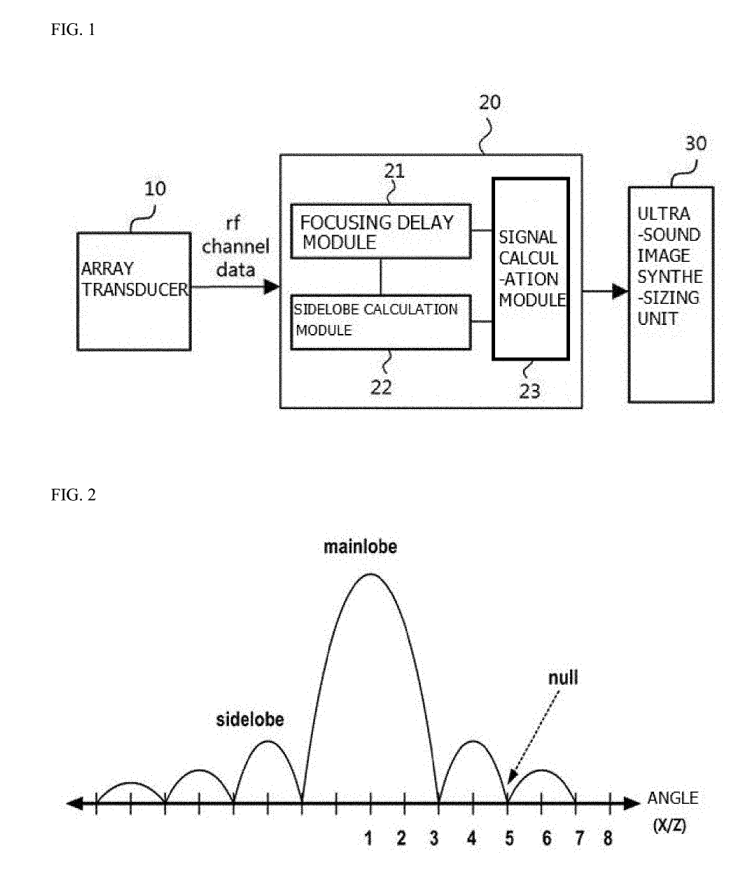

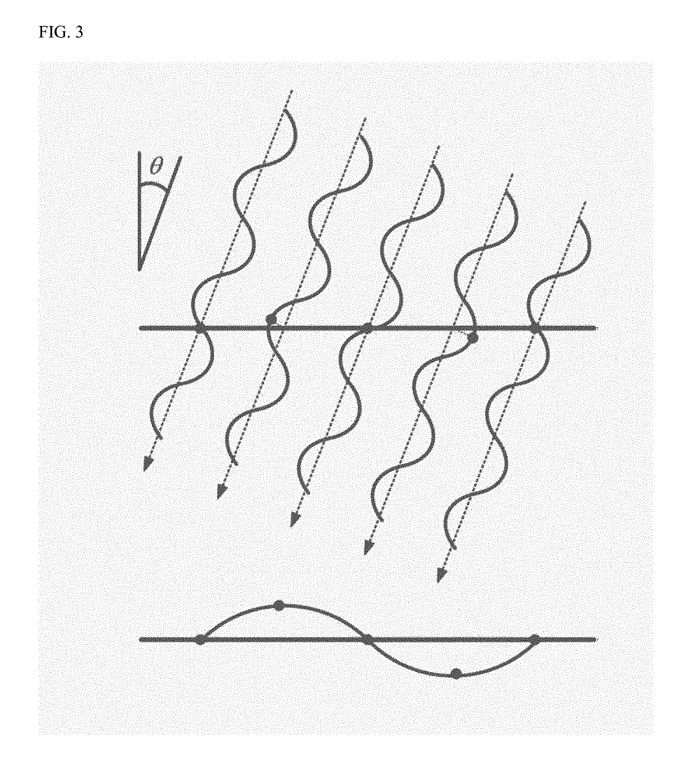

FIG. 1 is a block diagram illustrating a configuration of an apparatus to which a method for reducing a sidelobe in an ultrasound image according to an embodiment of the present invention is applied, and FIG. 2 is a view illustrating a sound field characteristic of an ultrasonic wave focusing system.

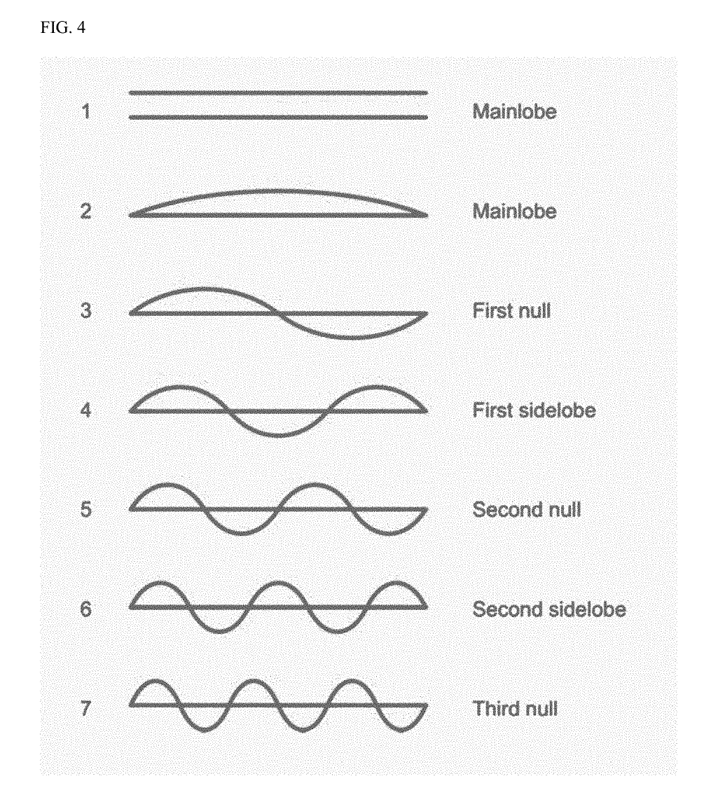

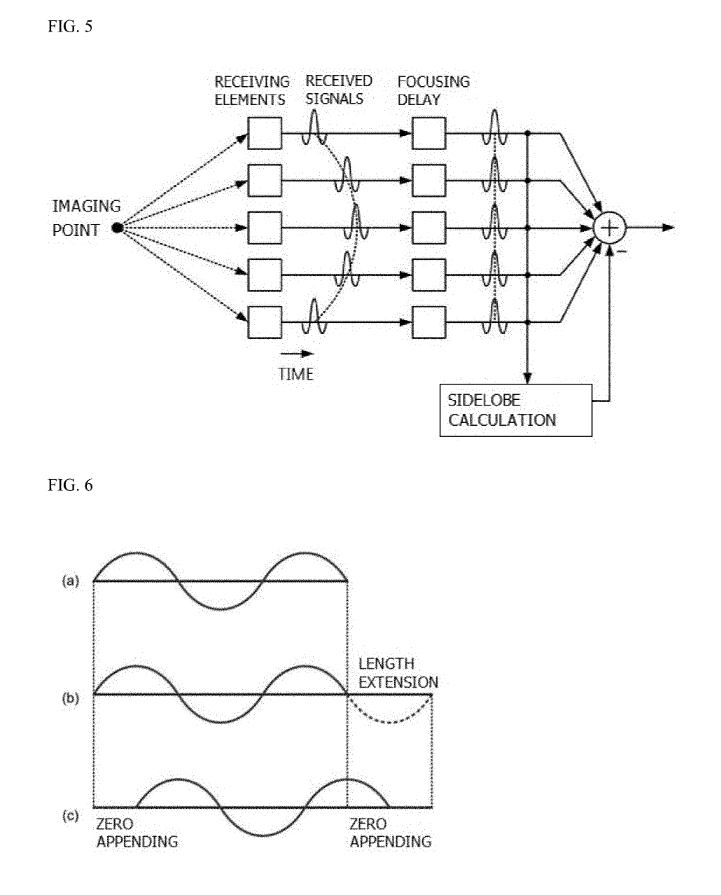

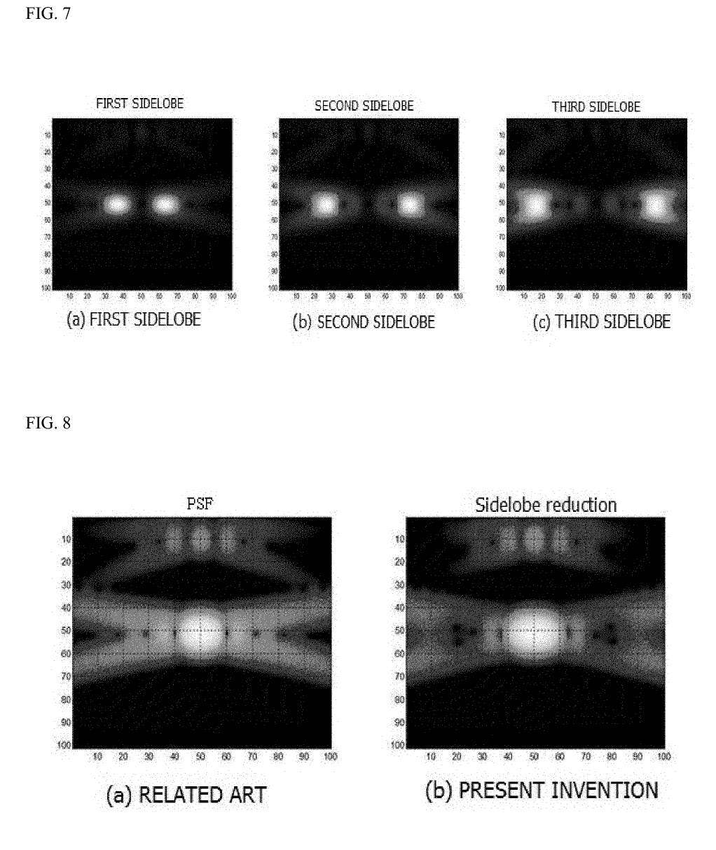

Also, FIG. 3 is a view for describing a principle of a sidelobe formation in a sound field of an ultrasonic wave focusing system, FIG. 4 is a view illustrating a waveform of signals across a transducer, which impinge on the transducer at a certain angle shown in FIG. 2, and FIGS. 5 and 6 are views for describing a calculation process for removing a sidelobe by an apparatus for reducing a sidelobe in an ultrasound image according to an embodiment of the present invention.

As illustrated in FIG. 1, an apparatus for reducing a sidelobe in an ultrasound image according to the present invention includes an array transducer 10, a receiving and focusing unit 20, and a unit image synthesizing unit 30.

The array transducer 10 is configured to transmit ultrasonic waves to an inside of a human body and have a plurality of receiving elements linearly arranged to receive signals reflected from a tissue of the human body. In the present embodiment, a case in which the number of receiving elements is 128 (i.e., a case in which the number of channels is 128) will be described as an example for convenience of description.

Also, the receiving and focusing unit 20 forms a scan line for forming an ultrasound image from channel signals received from each channel (i.e., receiving element) of the array transducer. For this, the receiving and focusing unit 20 includes a focusing delay module 21, a sidelobe calculation module 22, and a signal calculation module 23.

A calculation process of the receiving and focusing unit 20 is illustrated in FIG. 5, and each step of the calculation process will be described in detail below.

First, as described above, signals reflected from a human body tissue (i.e., an imaging point) arrive at each of the receiving elements at different times due to positions at which the receiving elements are arranged. The focusing delay module 21 applies a time delay to each of a plurality of channel signals in which a difference in arrival time has occurred as above to temporally align the channel signals as if the channel signals had arrived at the same time.

Also, the sidelobe calculation module 22 serves to calculate a frequency component (i.e., a waveform, a magnitude, or both of a sidelobe signal) of sidelobe signal components included in the delayed channel signals for focusing by a method to be described below.

As illustrated in FIG. 2, a sound field characteristic obtained from an image region of a general ultrasonic wave focusing system is that a mainlobe is formed with respect to a scan line direction of a transducer and sidelobes are formed at both sides of the mainlobe due to a leakage of ultrasonic signals.

In this manner, when the signals impinge on the transducer from directions adjacent to and at random angles with the scan line direction of the transducer, the signals are incident on the receiving elements with different phases as illustrated in FIG. 3.

Consequently, the signals incident at the random incident angles are shown as signals having a specific frequency referred to as a spatial frequency when viewed in the transducer. The spatial frequency may be expressed as [Equation 1] below and may vary according to a direction in which a sidelobe is formed.

$$f = \frac{D}{λ} sin θ \qquad [ Equation 1 ]$$

Here, in [Equation 1] above, D represents a size of a transducer, λ represents the wavelength of a center frequency of an ultrasonic wave, and θ represents an incident angle of an ultrasonic signal with respect to a scan line.

Meanwhile, FIG. 4 illustrates waveforms of ultrasonic signals incident on a transducer at angles corresponding to numbers on a horizontal axis in the sound field characteristic of the ultrasonic wave focusing system illustrated in FIG. 2. It can be recognized that signals incident at angles corresponding to numbers 1 and 2 are signals forming the mainlobe.

Also, it can be recognized that, among signals shown at both sides of the mainlobe, signals incident at angles corresponding to odd numbers (3, 5, 7 . . . ) (hereinafter, referred to as “null direction”) have an integer number of cycles whereas the signals incident at angles corresponding to even numbers (4, 6, 8 . . . ) (hereinafter, referred to as “sidelobe direction”) have a non-integer number of cycles.

Here, because signals incident in the null direction are signals whose spatial frequency has an integer as CPA and the number of cycles thereof is an integer as described above, the signals are shown as zero in a process of adding up focusing-delayed channel signals and do not form sidelobe noise as will be described below.

However, because signals incident in the sidelobe direction are signals whose spatial frequency has (integer+0.5) as CPA and the number of cycles thereof is a non-integer as described above, half cycle components are not removed in a process of adding up focusing-delayed channel signals and thus act as sidelobe noise in an image as will be described below.

Consequently, it can be recognized that sidelobe components can be removed from an ultrasound image when, among channel signals received by the transducer, signal components having a spatial frequency forming a sidelobe (the spatial frequency is 1.5 CPA in a case of a first sidelobe component, the spatial frequency is 2.5 CPA in a case of a second sidelobe component, etc.) are removed.

The present invention uses the above-described sidelobe formation principle to remove a sidelobe component from an ultrasound image. Specifically, already known spatial frequencies of sidelobe components (i.e., 1.5 CPA, 2.5 CPA, etc.) and the number of channels (i.e., the number of receiving elements) are used to calculate frequency components of sidelobe signals having the corresponding spatial frequencies, and the frequency components of the sidelobe signals are subtracted in a process of adding up the focusing-delayed channel signals, thereby removing sidelobe components from an ultrasound image.

Here, the calculating of the frequency components of the sidelobe signals includes obtaining a waveform, a magnitude, or both of the corresponding sidelobe signals. In the present embodiment, as an example, a process of calculating the frequency components includes calculating a waveform of a corresponding sidelobe signal and then calculating a magnitude of the corresponding sidelobe signal from the calculated waveform thereof.

For this, the sidelobe calculation module 22 calculates a magnitude of a sidelobe component signal by estimating a frequency of a sidelobe signal using the following method.

Generally, a waveform or a magnitude of a signal received by a transducer may be calculated using any known frequency estimation techniques such as the Fourier transform. Such frequency estimation methods can accurately calculate a magnitude of a frequency component that corresponds to an integer multiple of a frequency that corresponds to an inverse of a signal length but cannot accurately calculate a magnitude of a frequency component that is not an integer multiple of the frequency due to the window effect caused by finite-length data.

Consequently, to solve this, the sidelobe calculation module 22 employs a method in which a length of a sidelobe signal corresponding to a sidelobe component is extended to a length at which frequency estimation is possible by calculation according to a predetermined method and the magnitude of the corresponding sidelobe signal having an extended signal length is calculated using a frequency estimation method such as the Fourier transform.

In this case, the sidelobe calculation module 22 extends a signal length of the corresponding sidelobe signal by appending zeros to a front end, a rear end, or both of the corresponding sidelobe signal so that the spatial frequency becomes a nearest positive integer in an extended length signal.

For example, in a case of a signal corresponding to the first sidelobe component illustrated in FIG. 2 or 4, because the signal has a spatial frequency of 1.5 CPA as illustrated in (a) of FIG. 6, a length of a channel signal becomes an integer-multiple frequency signal when the length of the signal is extended and the spatial frequency becomes 2 CPA as illustrated in (b) of FIG. 6. Thus, a frequency component of the spatial frequency, 2 CPA, can be accurately calculated by a frequency estimation method such as the Fourier transform.

Also, because a length of an actually received signal is D (i.e., the number of receiving elements or the number of channels), the sidelobe calculation module 22 may extend a signal length of a channel signal corresponding to the first sidelobe component by [Equation 2] below.

$${D}_{Extended signal length} = \frac{2}{1.5} {D}_{Received signal length} \qquad [ Equation 2 ]$$

In this case, as it can be recognized from (c) of FIG. 6, the sidelobe calculation module 22 matches the extended signal length by appending zeros to a front end, a rear end, or both of the signal (appends zeros to both the front and rear ends as an example in the case of the present embodiment).

Meanwhile, in the case of the second sidelobe signal, likewise, the sidelobe calculation module 22 uses the known spatial frequency of the second sidelobe signal (2.5 CPA) and a received signal length or the number D of receiving elements to extend a length of the second sidelobe signal to a length at which frequency estimation is possible. In this case, the extended signal length has a value of 3D/2.5.

When the length of each of the sidelobe signals is extended according to the above-mentioned method, the sidelobe calculation module 22 can calculate a frequency component of the corresponding sidelobe components (i.e., a waveform, a magnitude, or both of a sidelobe signal) using a frequency estimation method such as the Fourier transform.

Also, the signal calculation module 23 forms a single scan line by subtracting the frequency components of each of the sidelobe signals calculated as described above in the process of adding up focusing-delayed channel signals and provides the scan line to the unit image synthesizing unit 30. In the case of the present embodiment, the signal calculation module 23 subtracts a magnitude of a corresponding sidelobe signal obtained by calculating a waveform of each of the sidelobe signals in the process of adding up the focusing-delayed channel signals.

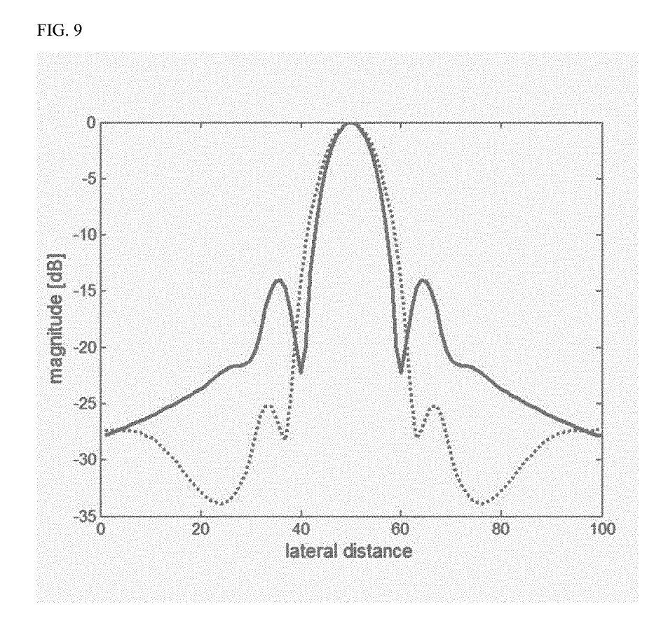

To verify the advantageous effects of the method for reducing a sidelobe in an ultrasound image according to the present invention configured as above, a transducer formed of 128 receiving elements and having a center frequency of 7.5 MHz was used to observe an imaging point at a depth of 35 mm when a transmit focal depth is 25 mm, and the results are illustrated in FIGS. 7 to 9.

As illustrated in FIG. 7, it can be recognized that the method according to the present invention accurately estimates a magnitude of a sidelobe component signal at a position at which a sidelobe is shown from a sound field characteristic of an ultrasonic wave focusing system.

Also, a point spread function (PSF) at an imaging point according to a method of the related art and a PSF at an imaging point according to the method of the present invention are compared and illustrated in FIG. 8. It can be recognized that sidelobes are removed and corresponding positions from which the sidelobes are removed are shown black in the case of (b) according to the present invention in which sidelobes are reduced.

Also, magnitudes of sidelobes at lateral positions of the PSFs of FIG. 8 at which sidelobes are present are compared in FIG. 9. The solid line indicates a characteristic of the lateral field response according to the method of the related art, and the dotted line indicates a characteristic of the lateral field response according to the method of the present invention.

As a result of the comparison, it can be confirmed that using the method of the present invention, sidelobes are reduced by 10 dB or more compared to the related art.

Meanwhile, because contrast resolution is decreased when a magnitude of a sidelobe is increased in an ultrasound image, a magnitude of a sidelobe calculated by the above-described method can be used as a metric for evaluating image quality.

Consequently, a quality factor (QF) for evaluating image quality of an ultrasound image is defined as a sum of magnitudes of sidelobe components as in [Equation 3] below in the present invention.

$$QF = {\sum }_{n = 1}^{K} {S}_{n} \qquad [ Equation 3 ]$$

Here, Sₙ represents a magnitude of a sidelobe component having an n^{th} spatial frequency. Although the QF can be obtained by adding up magnitudes of all sidelobe components theoretically, the QF may also be obtained by a sum of magnitudes of sidelobe components that have a significant influence on an ultrasound image (as an example, a sum of magnitudes of up to fifth sidelobe components) as needed.

Also, although a method for reducing a sidelobe in an ultrasound image by subtracting magnitudes of sidelobe components calculated by the sidelobe calculation module 22 in a process of adding up focusing-delayed channel signals by the signal calculation module 23 has been described in the case of the above-described embodiment, another embodiment of the present invention may be configured to reduce sidelobes in an ultrasound image by using a QF calculated as in [Equation 3].

In this case, the ultrasound image synthesizing unit 30 may be configured such that a brightness value of a pixel to be processed in an ultrasound image is changed inversely proportional to the QF in a process of synthesizing signals. Specifically, because the QF is proportional to a magnitude of a sidelobe, an ultrasound image is synthesized so that a brightness value of a corresponding pixel is decreased as the QF is increased.

For this, as an example, the present embodiment is configured to apply a weighting value to an ultrasound image being synthesized according to a predetermined method (or an expression) so that a brightness vale of each pixel forming the ultrasound image is inversely proportional to a QF of the corresponding pixel.

As described in detail above, the method for reducing a sidelobe in an ultrasound image according to the present invention is configured such that channel signals are obtained by applying focusing delays to signals received by receiving elements and then a known spatial frequency of a sidelobe signal and the number of channels are used to calculate a magnitude of the corresponding sidelobe signal, and an influence from the sidelobe signal is reduced by removing sidelobe components included in the channel signals by subtracting the calculated magnitude of the sidelobe signal in a process of adding up the focusing-delayed channel signals or by assigning a weighting value to an ultrasound image using the calculated magnitude of the sidelobe signal when synthesizing the ultrasound image.

Consequently, compared to the related art, the method for reducing a sidelobe in an ultrasound image has an advantage of being able to easily improve image quality of an ultrasound image because a magnitude of a sidelobe signal that affects image quality of an ultrasound image can be accurately calculated even with a small amount of calculation and the calculation result is applied to synthesis of the ultrasound image.

### INDUSTRIAL APPLICABILITY

The present invention is applicable to an image system using ultrasonic waves, particularly, to a medical ultrasound image system used in diagnosing lesions.

1. A method for reducing a sidelobe in an ultrasound image, the method comprising:
Step 1 of receiving, from individual receiving elements of an array transducer, ultrasonic signals reflected from an imaging point, and outputting the ultrasonic signals as channel signals of the corresponding receiving elements;
Step 2 of applying focusing delays to each of the channel signals to temporally align the channel signals; and
Step 3 of synthesizing an ultrasound image by using an added-up signal which is obtained by adding up the temporally aligned channel signals,
wherein Step 3 includes calculating a magnitude of a corresponding sidelobe signal in channel signals by using a spatial frequency of the sidelobe signal which generates a sidelobe and the number of receiving elements and synthesizing the sidelobe suppressed ultrasound image by subtracting the calculated magnitude of the sidelobe signal from the added-up signal.

2. The method of claim 1, wherein the calculating of the magnitude of the sidelobe signal in the temporally aligned channel signals includes:
Step 3-1 of extending a signal length of the sidelobe signal according to a predetermined method; and
Step 3-2 of estimating a waveform of the sidelobe signal having the extended signal length to calculate a frequency component of the corresponding sidelobe signal.

3. The method of claim 2, wherein:
the sidelobe signal is a signal whose spatial frequency is (positive integer+0.5) cycles per aperture (CPA) in an original channel signal; and
Step 3-1 includes extending a signal length of the corresponding sidelobe signal by appending zeros to a front end, a rear end, or both of the corresponding sidelobe signal so that the spatial frequency becomes a nearest positive integer in an extended length signal.

4. A method for reducing a sidelobe in an ultrasound image, the method comprising:
Step 1 of receiving, from individual receiving elements of an array transducer, ultrasonic signals reflected from an imaging point, and outputting the ultrasonic signals as channel signals of the corresponding receiving elements;
Step 2 of applying focusing delays to each of the channel signals to temporally align the channel signals; and
Step 3 of synthesizing an ultrasound image by using an added-up signal which is obtained by adding up the temporally aligned channel signals,
wherein Step 3 includes calculating a magnitude of a corresponding sidelobe signal by using a spatial frequency of the sidelobe signal which generates a sidelobe and the number of receiving elements and synthesizing the ultrasound image so that a brightness value of the ultrasound image is inversely proportional to the calculated magnitude of the sidelobe signal.

5. The method of claim 4, wherein:
the magnitude of the sidelobe signal is a quality factor (QF) value which is obtained by adding up magnitudes of a plurality of sidelobe signals having different spatial frequencies; and
Step 3 includes synthesizing the ultrasound image so that the brightness value of the ultrasound image is inversely proportional to the QF value.
